(19) United States
(12) Patent Application Publication
(10) Pub. No.: US 2024/0324859 A1
(43) Pub. Date: Oct. 3, 2024

(54) SYSTEM AND METHOD FOR STEREOSCOPIC IMAGE GENERATION

(71) Applicant: NEW YORK UNIVERSITY, New York, NY (US)

(72) Inventors: Eric Oermann, Chapel Hill, NC (US); Douglas Kondziolka, New York, NY (US); Kyunghyun Cho, New York, NY (US); Annika Brundyn, New York, NY (US); Jesse Swanson, New York, NY (US)

(21) Appl. No.: 18/690,480

(22) PCT Filed: Sep. 8, 2022
(86) PCT No.: PCT/US22/76101

§ 371 Date: Mar. 8, 2024

Related U.S. Application Data
(60) Provisional application No. 63/241,649, filed on Sep. 8, 2021.

(51) Int. Cl. A61B 1/00; A61B 1/05; H04N 13/156; H04N 13/261; H04N 23/50

(52) U.S. Cl. CPC A61B 1/00193; A61B 1/000096; A61B 1/00045; A61B 1/05; H04N 13/156; H04N 13/261; H04N 23/555

(57) ABSTRACT

A system for generating a target image comprises an endoscope having an image collection component, a computing device communicatively connected to the image collection component of the endoscope, comprising a non-transitory computer-readable medium with instructions stored thereon, which when executed by a processor perform steps comprising receiving at least one input image from the image collection component of the endoscope, providing the at least one input image as an input to a machine learning algorithm, generating a target image from the at least one input image using the machine learning algorithm, and providing the at least one input image and the target image to a display driver, and a display device, communicatively connected to the computing device, and configured to display the images provided to the display driver. A method of training a machine learning algorithm and a method of generating a stereoscopic image are also disclosed.

### CROSS-REFERENCE TO RELATED APPLICATIONS

This application is a 35 U.S.C. § 371 national phase application of International Application No. PCT/US2022/076101, filed Sep. 8, 2022, which claims priority to U.S. Provisional Patent Application No. 63/241,649 filed Sep. 8, 2021, both of which are incorporated herein by reference in their entirety.

### BACKGROUND OF THE INVENTION

The development and use of endoscopes for minimally invasive surgery is one of the major surgical innovations of the 20th century. An endoscope is a thin, tubular instrument with a camera attached to it. During surgery, the endoscope is inserted through a small incision or natural orifice, and the surgeon is then able to view the affected area digitally and operate on it by way of tiny surgical instruments (see FIG. 1). This obviates the need for large incisions, reducing patient healing time and the risk of post-operative infection. Technological advances in optics, illumination, and miniaturization over the past hundred years have expanded the reach of endoscopy, which has in turn revolutionized surgery.

While modern 3D endoscopes exist, due to the need to accommodate stereoscopic optics they are unable to be miniaturized to the same degree as 2D monoscopic endoscopes. Therefore, due to their larger size, stereoscopic endoscopes are not suitable for all types of surgery. Additionally, 3D endoscopes suffer from a reduced field of view relative to 2D endoscopes. The combination of a smaller field of view and lack of depth perception can be particularly challenging when operating around critical neurological structures.

A major drawback of early endoscopy was the lack of depth perception arising from the two-dimensional camera view. Although quite a few surgeries can safely be performed using the 2D view, it is more challenging to operate around critical and highly complex physiological structures in this format. It is difficult for a surgeon to see for instance how close a brain tumor is to the surrounding nerves and tissue without depth perception. Such difficulty is naturally resolved with 3D imaging. Furthermore, 3D visualization has a shallower learning curve and can help novice surgeons take advantage of minimally invasive techniques. While modern 3D endoscopes exist, they cannot be miniaturized to the same degree as 2D endoscopes have been (4 mm vs 2.7 mm in diameter for the smallest in each class). They also suffer from a smaller field of view, rendering them infeasible for some types of surgeries.

3D content is typically stored in stereo format: i.e., two perspectives of the same scene. When viewed together, the disparity between the two perspectives simulates natural binocular vision, resulting in a 3D experience. In the absence of a 3D endoscope with two cameras, one can generate an alternate view of the existing 2D input, which, combined with the original view, forms a stereo pair. The result can be viewed using 3D glasses or a head-mounted VR display (see FIG. 1) effectively recreating 3D perception for a surgeon during surgery.

2D to 3D (monoscopic to stereoscopic) video conversion is a classical computer vision problem. 3D videos are often stored in a stereoscopic format. For each frame, the format contains two projections of the same scene; one for the viewer's left eye and one for the viewer's right eye. The disparities of the two perspectives simulate natural binocular vision resulting in a 3D experience. Solving this problem of converting 2D to 3D video entails reasoning about depth from a single perspective and synthesizing a novel view for the other eye. This presents a highly under-constrained problem. In addition to depth ambiguities, some pixels in the novel view correspond to geometry that is not currently visible from the current perspective. The missing data must be hallucinated by the model or generated from previous different perspectives of the same scene.

Traditional 2D-to-3D reconstruction methods often consist of two stages. First, a depth map is constructed from the 2D input; then, a depth image-based rendering (DIBR) algorithm combines the depth map with the input view to generate the missing view of the stereo pair. Depth maps can be constructed using various techniques, among them manual construction by artists, structure-from-motion (SfM) approaches, and, more recently, machine learning algorithms.

Monocular depth estimation, however, is in itself a difficult task. Recent trends in deep learning have instead shifted towards training an end-to-end differentiable system, bypassing depth estimation.

Stereoscopic 3D visualization in medical endoscopic teleoperation results in improved user performance in some tasks when compared to monoscopic endoscopy. Therefore, there is a need in the art for a system and method of stereoscopic 3D generation from a monoscopic 2D endoscopic view, in order to improve user performance for surgical tasks.

### SUMMARY OF THE INVENTION

In one aspect, a system for generating a target image comprises an endoscope having an image collection component, a computing device communicatively connected to the image collection component of the endoscope, comprising a non-transitory computer-readable medium with instructions stored thereon, which when executed by a processor perform steps comprising receiving at least one input image from the image collection component of the endoscope, providing the at least one input image as an input to a machine learning algorithm, generating a target image from the at least one input image using the machine learning algorithm, and providing the at least one input image and the target image to a display driver, and a display device, communicatively connected to the computing device, and configured to display the images provided to the display driver.

In one embodiment, the at least one input image comprises a sequence of at least five frames of a video recorded by the image collection component. In one embodiment, the image collection component is a camera. In one embodiment, the endoscope further comprises a tube with the image collection component positioned at a distal end of the tube, the tube having an outer diameter of at most 10 mm. In one embodiment, the computing device is positioned in the display device. In one embodiment, the computing device is positioned in the endoscope. In one embodiment, the machine learning algorithm is selected from a convolutional neural network, a generative/adversarial neural network, or a U-Net.

In one embodiment, the steps further comprise buffering a sequence of input images to process with the machine learning algorithm. In one embodiment, the sequence comprises at least five input images.

In one aspect, a method of training a machine learning algorithm for 3D reconstruction comprises providing a set of rectified stereo video frames, selecting a training subset of the set of rectified stereo video frames and isolating one view from each of the selected stereo video frames, providing a sequence comprising at least one input video frame from the isolated view to a machine learning algorithm to generate a target frame corresponding to the at least one input video frame, calculating a loss function value from the generated target frame by comparing it to the known corresponding video frame from the set of stereo video frames, and adjusting at least one parameter of the machine learning algorithm based on the calculated value of the loss function. In one embodiment, the sequence comprises at least five video frames.

In one embodiment, the machine learning algorithm is selected from a convolutional neural network, a deep neural network, a U-Net, or a generative/adversarial neural network. In one embodiment, the loss function is selected from mean-squared error, least absolute deviations, least square errors, or perceptual loss function. In one embodiment, the machine learning algorithm comprises an automated metric selected from Learned Perceptual Image Patch Similarity, Deep Image Structure and Texture Similarity, Fréchet Inception Distance, Peak signal-to-noise ratio, or Structural Similarity Index. In one embodiment, the automated metric is selected from Learned Perceptual Image Patch Similarity and Deep Image Structure and Texture Similarity.

In one aspect, a method of generating a stereoscopic image for a user of an endoscope comprises receiving at least one input image from an image collection component of an endoscope, providing the at least one input image as an input to a machine learning algorithm, generating a target image from the at least one input image using the machine learning algorithm, and displaying the at least one input image and the target image on a display device as a stereoscopic image.

In one embodiment, the at least one input image comprises a sequence of at least five frames of a video recorded by the image collection component. In one embodiment, the machine learning algorithm is selected from a convolutional neural network, a generative/adversarial neural network, or a U-Net. In one embodiment, the method further comprises buffering a sequence of input images to process with the machine learning algorithm. In one embodiment, the sequence comprises at least five input images. In one embodiment, the method further comprises upsampling the at least one input image using bilinear interpolation or strided transpose convolution.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a schematic view of an exemplary method of stereoscopic image generation;

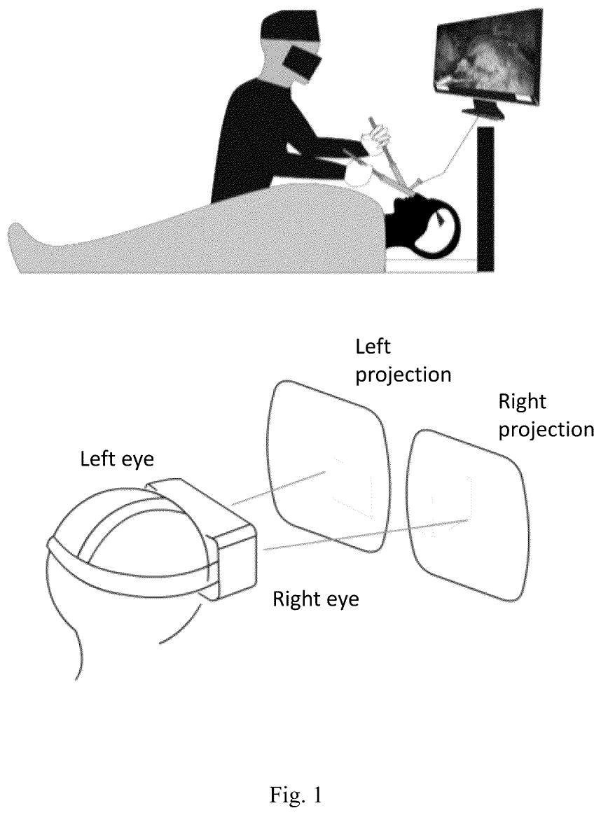

FIG. 2 is an exemplary computing device;

FIG. 3 is an exemplary stereoscopic image generation device;

FIG. 4 is a method of training a machine learning algorithm;

FIG. 5 is a schematic view of a method of stereoscopic imaging;

FIG. 6 is a graphical illustration of a processing method of the disclosure;

FIG. 7 is a graphical illustration of a method of stereoscopic image generation;

FIG. 8 is a set of exemplary input and target images;

FIG. 9 is a diagram of an exemplary two-alternative forced choice test;

FIG. 10 is a set of exemplary input and target images;

FIG. 11 is a graphical depiction of image artifacts;

FIG. 12 is a set of exemplary input and target images;

FIG. 13 is a set of exemplary generated images; and

FIG. 14 is a target image and a set of exemplary generated images.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C #, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

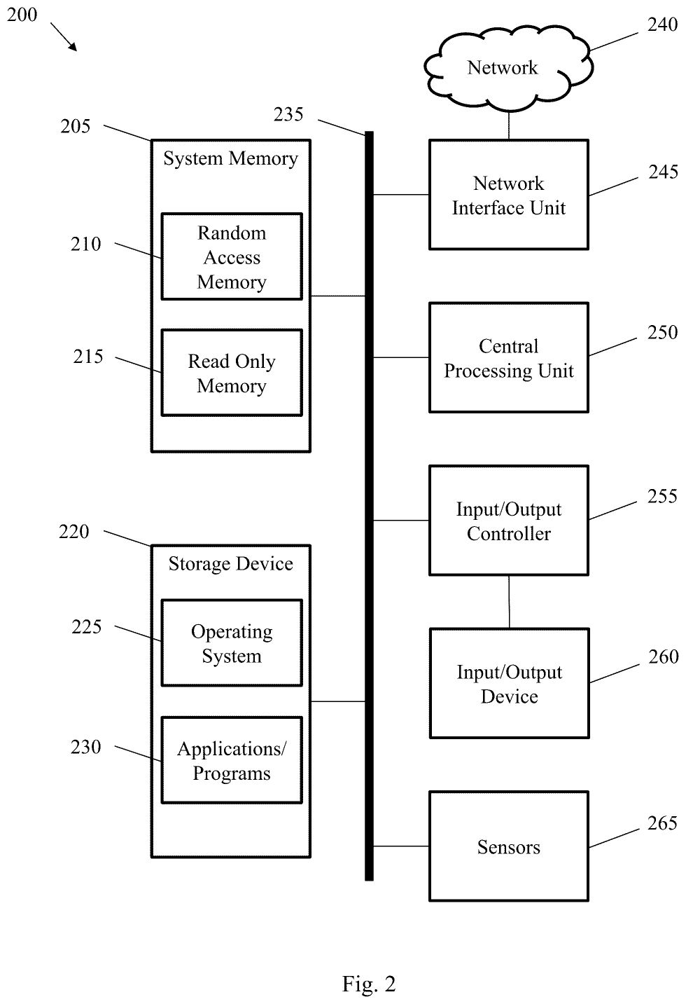

FIG. 2 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 2 depicts an illustrative computer architecture for a computer 200 for practicing the various embodiments of the invention. The computer architecture shown in FIG. 2 illustrates a conventional personal computer, including a central processing unit 250 (“CPU”), a system memory 205, including a random access memory 210 (“RAM”) and a read-only memory (“ROM”) 215, and a system bus 235 that couples the system memory 205 to the CPU 250. A basic input/output system containing the basic routines that help to transfer information between elements within the computer, such as during startup, is stored in the ROM 215. The computer 200 further includes a storage device 220 for storing an operating system 225, application/program 230, and data.

The storage device 220 is connected to the CPU 250 through a storage controller (not shown) connected to the bus 235. The storage device 220 and its associated computer-readable media provide non-volatile storage for the computer 200. Although the description of computer-readable media contained herein refers to a storage device, such as a hard disk or CD-ROM drive, it should be appreciated by those skilled in the art that computer-readable media can be any available media that can be accessed by the computer 200.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 200 may operate in a networked environment using logical connections to remote computers through a network 240, such as TCP/IP network such as the Internet or an intranet. The computer 200 may connect to the network 240 through a network interface unit 245 connected to the bus 235. It should be appreciated that the network interface unit 245 may also be utilized to connect to other types of networks and remote computer systems.

The computer 200 may also include an input/output controller 255 for receiving and processing input from a number of input/output devices 260, including a keyboard, a mouse, a touchscreen, a camera, a microphone, a controller, a joystick, or other type of input device. Similarly, the input/output controller 255 may provide output to a display screen, a printer, a speaker, or other type of output device. The computer 200 can connect to the input/output device 260 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 220 and/or RAM 210 of the computer 200, including an operating system 225 suitable for controlling the operation of a networked computer. The storage device 220 and RAM 210 may also store one or more applications/programs 230. In particular, the storage device 220 and RAM 210 may store an application/program 230 for providing a variety of functionalities to a user. For instance, the application/program 230 may comprise many types of programs such as a word processing application, a spreadsheet application, a desktop publishing application, a database application, a gaming application, internet browsing application, electronic mail application, messaging application, and the like. According to an embodiment of the present invention, the application/program 230 comprises a multiple functionality software application for providing word processing functionality, slide presentation functionality, spreadsheet functionality, database functionality and the like.

The computer 200 in some embodiments can include a variety of sensors 265 for monitoring the environment surrounding and the environment internal to the computer 200. These sensors 265 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

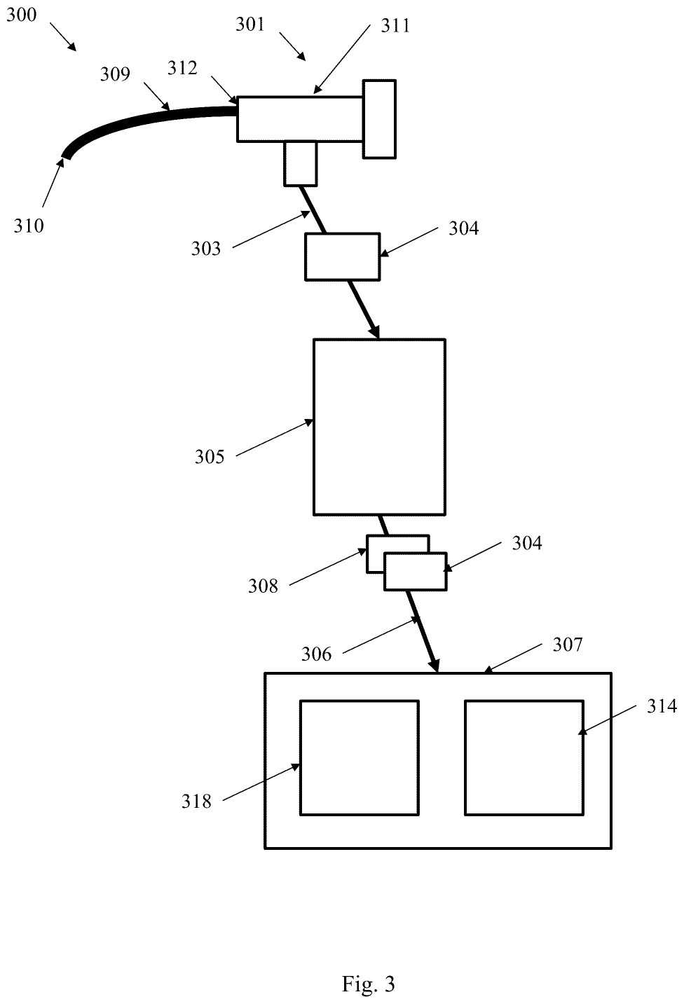

One embodiment of a system 300 of the invention is shown in FIG. 3. The system 300 includes an endoscope 301 which in some embodiments is a monoscopic endoscope. Endoscope 301 may include a single camera 310, which may for example be positioned at a distal end of a tube 309, with a data connection configured to transmit image data from the camera 310 to a controller in the housing 311 of the endoscope 301. In some embodiments the distal end of the tube 309 further includes an illumination element, for example an LED, configured to illuminate the field of view of the camera 310. In some embodiments, the illumination element may be positioned at the proximal end 312 of the tube 309, with the light generated by the illumination element transmitted to the distal end of the tube 309 for example via one or more optical fibers. Although stereoscopic endoscopes, with two cameras positioned at the distal end of the tube 309, exist, the increased thickness necessary at the distal end of tube 309 means that such endoscopes are not well suited for all procedures, for example neuro-endoscopic procedures. In some embodiments, the tube 309 of endoscope 301 may have a maximum outer diameter of no more than 1 mm, no more than 2 mm, no more than 3 mm, no more than 4 mm, 5 mm, no more than 6 mm, no more than 7 mm, no more than 7.5 mm, no more than 8 mm, no more than 8.5 mm, no more than 9 mm, no more than 9.5 mm, or no more than 10 mm.

The endoscope 301 is communicatively connected to computing device 305 via data link 303, which may be any wired or wireless data link known in the art. The data link in some embodiments transmits a sequence of images or a video stream 304 from camera 310. Computing device 305 performs processing steps on the image sequence or video stream 304 as disclosed herein, using the image sequence or video stream 304 as an input stream comprising one or more input images. The processing steps may take as an input one, two, three, four, five, six, seven, eight, nine, ten, or more input frames from the input stream in order to generate one or more target frames. The target frames are then assembled sequentially into a companion target image sequence or video stream, and the input stream 304 and target stream 308 are transmitted from computing device 305 along second data link 306 to display device 307, which may in some embodiments be a stereoscopic display device. Display device 307 may then display the input image sequence or video stream 304 on a first display 314 for one eye of a user, and the target image sequence or video stream 308 on a second display 318 for a second eye of the user. The effect of the two displays is an artificial stereoscopic view of the region in the field of view of the endoscope camera 310 which includes one true view transmitted from the camera and a second inferred view displayed for the user's second eye, in order to add depth to the image for the user and allowing the user to maneuver the endoscope hose 309 more precisely within a patient.

Like the first data link 303, the second data link 306 may be any wired or wireless connection known in the art or contemplated herein. Computing device 305 may similarly be any suitable computing device, including but not limited to a laptop, desktop, tablet, smartphone, or embedded microcontroller. In some embodiments, computing device 305 may be physically incorporated into the housing 311 of endoscope 301, or into display device 307.

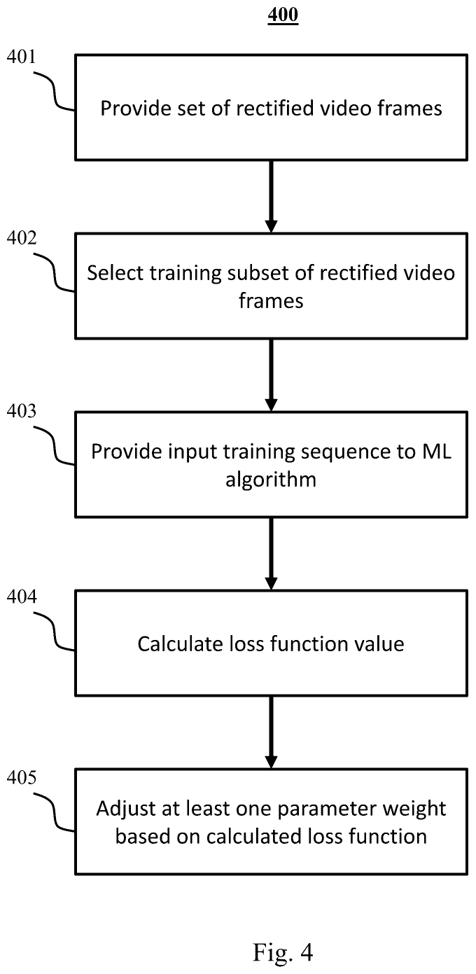

In one aspect, a method of training a machine learning algorithm to generate a stereoscopic image is disclosed, as shown in FIG. 4. The method includes the step of providing a set of rectified stereo video frames in step 401, selecting a subset of the set of rectified stereo video frames and isolating one view from each of the selected stereo video frames in step 402, providing a sequence comprising at least one input video frame from the isolated view to a machine learning algorithm to generate a target frame corresponding to at least one of the input video frames in step 403, calculating a loss function value from the generated target frame by comparing it to the known corresponding video frame from the set of rectified video frames in step 404, and adjusting at least one parameter weight based on the calculated loss function in step 405. In some embodiments, once trained, the machine learning algorithm may be used in computing device 305 of FIG. 3 in order to calculate the target image sequence or video stream 308 from the input image sequence or video stream 304.

Any suitable machine learning algorithm may be used, including but not limited to a neural network, a convolutional neural network, a deep neural network, a U-Net, a generative/adversarial neural network, or any general encoder/decoder framework that has as its output stereoscopic imaging.

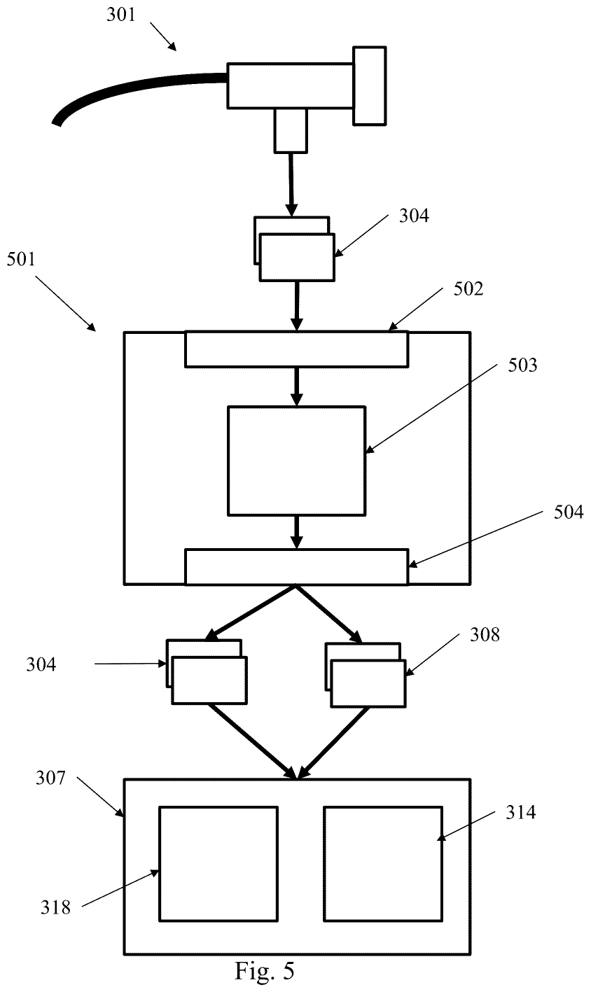

A schematic view of a method of the disclosure is shown in FIG. 5. Input images 304 are first gathered by endoscope 301. Endoscope 301 is in some embodiments a monoscopic endoscope as discussed above, having only a single image collection component, for example a camera. Input images 304 may comprise a single image at a time, or may alternatively comprise a sequence of input images, for example consecutive frames or non-consecutive frames in a video stream recorded from the image collection component in the endoscope 301. The images may have any suitable resolution, including but not limited to 384×192, 320×240, 640×480, 720×480, 768×384, 800×600, 1024×1168, 1024×720, 1280×1024, 1280×720, 1920×1080. As would be understood by one skilled in the art, in some embodiments the image may have a resolution that is arbitrarily large. The video frame or frames may then be provided to an input frame buffer 502 of an image generation program 501. In some embodiments, input frame buffer 502 may collect 2, 3, 4, 5, 6, 7, 8, 9, 10, or more frames in order to generate a target frame. Image generation program 501 may comprise image generation engine 503, which may in some embodiments comprise or be a machine learning algorithm as disclosed herein. The image generation engine 503 may be configured to generate target images 308 from one or more of the input images 304 stored in input frame buffer 502. In some embodiments, the image generation engine 503 is configured to store one or more target images 308 in an output frame buffer 504, but in other embodiments the image generation engine provides unbuffered output to an output display device, for example stereoscopic viewer 307 having first and second displays 314 and 318 for displaying the input and target images. In some embodiments, an input image may be displayed on a left eye display and a target image may be displayed on a right eye display, but in other embodiments the input image may be displayed on the right eye display and the target image may be displayed on the left eye display.

In some embodiments, the input and target image may be swappable during operation by a user, for example in some embodiments where an image collection component of an endoscope may become positioned in an orientation that is not optimal for generating a stereoscopic view in one direction, the user may toggle which eye is generated and which eye is the input image in order to provide a more useful perspective.

In some embodiments, only the image data (comprising one or more frames at a time) from a single image collection component is provided to the image generation program in order to generate a target image. In other embodiments, the image data may be supplemented, for example with depth information (e.g. via an ultrasonic or other depth sensor positioned in the endoscope tip) or transformed medical image information, for example x-ray, CT-scan, MM, or other medical image data which may be used to approximate the size and distance of various organs in the area being observed by the endoscope.

Stereo-vision video reconstruction is the task of generating a second video stream projection of a scene for a second eye given a single monoscopic video stream for a first eye in a way that simulates binocular stereo vision when the two streams are viewed together. Stereo-vision methods usually exploit epipolar geometry to estimate disparity maps. Disparity maps represent the difference, or disparity, of an object in two rectified images of the same scene. In the simplest model, two identical coplanar cameras with focal length f are separated a baseline distance b along the x dimension. The disparity of a point in the scene in the image plane is given by:

$$d = {x}_{l}^{′} - {x}_{r}^{′} = \frac{b ( z - f )}{z} \qquad Equation 1$$

If one can estimate the quantity b(z−f)/z, is possible to shift the corresponding x′ₗ in the left image, I_{L}, by d to generate the corresponding point in the right image I_{R}. With this approach, occluded parts of the input image must be inpainted to generate a complete image. Furthermore, if the problem is constrained such that the dimensions of I_{L}==I_{R}, the rightmost parts of I_{R} must be inpainted because parts of the image are not present in I_{L}. Disclosed herein is an end-to-end pipeline for monoscopic 2D to 3D stereoscopic video reconstruction that directly regresses on right images to implicitly learn disparity, inpainting, and other transformations required for the task of stereo reconstruction.

Disclosed herein is a system and method for generating a stereoscopic 3D video from a monoscopic 2D video source, which may in some embodiments be a 2D endoscopic view.

The tasks and the approaches disclosed herein are not restricted to surgical endoscopy and are generally applicable to any video. The endoscopy use case in particular is interesting and meaningful technically, as factors such as sharpness, hallucinations, and accurate depth perception are more salient than in many other applications. It is anticipated that the findings from this work will be applicable and transferable to other similar problems in the future.

In one embodiment, the system uses a deep neural network (DNN). In this approach, a deep neural network is trained to use the information available in past video frames to reconstruct an alternative perspective of the current frame. This may be necessary in some embodiments because it is highly under-constrained to create an alternative perspective from a single view. For instance, some regions are simply invisible in the current view and cannot be reconstructed from one view alone. Past frames may however contain information about such occluded regions and objects, enabling more accurate depth estimation.

Test of a series of variants of deep neural networks are disclosed in the below Experimental Examples to confirm this hypothesis (multiple past frames to facilitate 3D reconstruction) and to identify an optimal network architecture and learning setup. Extensive and rigorous evaluation of these variants of deep neural networks were conducted by two sets of reader studies involving experienced surgeons. The reader studies confirm the importance of using multiple past frames and also the effectiveness of a properly designed deep neural network in benefiting from these past frames. In doing so, a diverse set of automated metrics were tested and correlated against the outcome of these reader studies, based on which two perceptual metrics, DISTS and LPIPS were identified as correlated most with expert judgement.

In one embodiment, the disclosed approach is similar to the one disclosed in Xie, et al., “Deep3d: Fully automatic 2d-to-3d video conversion with deep convolutional neural networks,” 2016. In one embodiment, the disclosed method uses a deep learning based approach to directly regress on ground truth target images. The approach is based on a fully-convolutional U-Net architecture. U-Nets have been used for the closely related task of depth estimation in existing works, for example in Wiles et al., “Synsin: End-to-end view synthesis from a single image,” 2020. U-Nets have also been adapted to video data for image segmentation tasks by including temporal modules in the encoder with skip connections to the decoder or as modules between at every skip connection level. Novel view synthesis with a U-Net architecture has been explored for large geometric changes. In their main experiments, Xie et al. focus on using a single RGB frame as input and find minor improvements when using optical flow or multiple frames as inputs while using Mean Absolute Error (MAE) was used as the primary metric for evaluation. As shown in the Experimental Examples below, the method of the present disclosure, which uses multiple frames as inputs to a machine learning model, results in superior quantitative and qualitative performance when compared to single frame models with equivalent architectures.

Furthermore, the disclosed method may in some embodiments be built as a fully convolutional encoder-decoder architecture, largely adapting the U-Net architecture. U-Nets have been used for the closely related task of depth estimation. U-Nets have also been applied to the task of semi-supervised video segmentation. Furthermore, novel-view synthesis with a U-Net architecture has been explored for large geometric changes.

Most of the existing work related to the present disclosure focuses on depth estimation and topographical reconstruction in endoscopy. For example, Kumar et al. (Stereoscopic visualization of laparoscope image using depth information from 3d model. Computer Methods and Programs in Biomedicine, 113(3):862-868, 2014, incorporated herein by reference).design a five-stage procedure, involving 3D shape reconstruction, registration with a 3D CT model, endoscope position tracking, depth map calculation, and stereo image synthesis. These stages are often developed and tuned separately from the other stages and require expensive ground-truth annotations.

Unlike these earlier studies, the present disclosure provides an end-to-end approach that requires minimal engineering and annotation.

It is tempting to generalize the success of deep learning for “natural” video to surgical video. It is however unclear whether this generalization is reasonable, since there are many features that are specific to surgical video and are not present in natural video. These include soft textures, homogeneous colors, inconsistent sharpness, variable depth of field and optical zoom, inconsistent motion, and obstructions arising from e.g. fluid and smoke. This discrepancy between natural and surgical video suggests that it is necessary to investigate the applicability and effectiveness of deep learning based end-to-end approaches to 2D-to-3D reconstruction in surgical video.

As discussed above, stereo video consists of two rectified views corresponding to a left and right view of a single scene. Throughout this disclosure, the left view is referred to interchangeably as the “input view,” and right view is referred to interchangeably as “the target view,” although it is understood that in some embodiments the right view may be used as the input view and the left view may be used as the target view. The task of stereo video reconstruction is defined in one embodiment of this disclosure as follows: First, let V_{L} define a given video stream of the input view with a sequence of T frames, V_{L}={I_{L,1},I_{L,2}, . . . , I_{L,T}}. The systems and methods disclosed herein generate a projection of the scene I_{R,t} corresponding to each input frame I_{L,t} in sequence V_{L}, for each time index t∈{1, . . . , T}. In addition to using the current frame I_{L,t} to generate the target frame I_{R,t}, the present disclosure further uses previous frames as context to improve the estimation of the target frame I_{R,t}. Therefore, the input to the disclosed model is generalized as follows: Let the input for the task be a sub-sequence of video frames with length K: x_{L,t,k}={I_{L,t-(k-1)}, . . . , I_{L,t-1},I_{L,t}}. The dimensions of the sub-sequence are x_{L,t,k} ∈R^{k×3×H×W} where k is the number of frames, H and W are the frame height and width, and 3 is the number of channels (RGB). t denotes the current frame and the preceding K−1 frames are context frames. x_{L,t,k} where K≥0 is then used to generate I_{R},t.

The disclosed methods build on the U-Net architecture disclosed in Ronneberger O., et al., (2015) U-Net: Convolutional Networks for Biomedical Image Segmentation. Medical Image Computing and Computer-Assisted Intervention—MICCAI 2015. MICCAI 2015. Lecture Notes in Computer Science, vol 9351.

The disclosed implementation differs from the original architecture in at least the following two ways. First, decoder blocks use padding instead of cropping to concatenate features from the skip connections and second, the decoder uses either bilinear interpolation or strided transpose convolution for upsampling. In other embodiments, any other means of upsampling may be used, including but not limited to quadratic interpolation, nearest neighbor interpolation, bicubic interpolation, or formal deconvolution.

Let g_{θ} denote a U-Net based fully-convolutional architecture used to generate the corresponding stereo view. The 2D to stereoscopic 3D conversion task is to predict a corresponding stereo right view for the current left frame at time t, yₜ=g_{θ}(x_{t,-K:t}).

In one embodiment, a single frame computation approach is disclosed wherein the current frame is used as input (K=1) to the network g_{θ}, and the system predicts the corresponding stereo (target) frame yₜ based only on the input frame.

In one embodiment, K>1 input frames are used, for example the current frame and the previous frame, the current frame and the previous two frames, or a set including the current frame and the previous 3, 4, 5, 6, 7, 8, 9, 10, 15, 20, 30, or 50 frames. Fully-convolutional networks are not designed to model temporal dependencies. However, in some embodiments, the temporal ordering in the video may be ignored and the K frames treated analogously to channels, or colors. In one embodiment, the input 4D tensor x_{≤t}∈ [symbol] ^{K×3×H×W} is reshaped into a 3D tensor of size 3K×H×W and the same U-Net architecture g_{θ} described above is applied, as if it were an image with 3×K color channels, to predict yₜ.

In one embodiment, a modified spatio-temporal U-Net is used. Like spatial structures, temporal structures exhibit themselves at multiple scales. Pixel-level temporal information can facilitate inferring fine-grained textures, while object-level temporal information can facilitate overcoming occlusion in individual frames. In order to better incorporate such multi-scale nature of temporal information, the U-Net architecture was modified by inserting a “temporal module” at each scale between the encoder and decoder, capturing unique aspects of temporal information at each scale. In one embodiment, a more advanced temporal module may be used, for example a recurrent neural net based module.

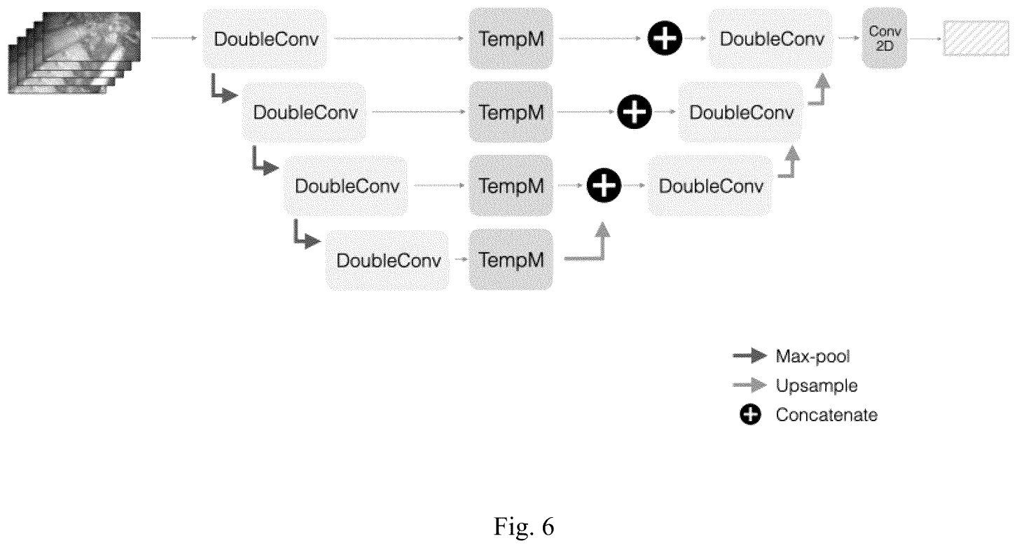

More specifically, each of K consecutive frames in the input x_{≤t} is fed to the encoder separately and results in K spatial feature maps. This sequence of feature maps is processed by the temporal module, before being sent to the corresponding layer at the decoder for concatenation. A graphical illustration of the method is in FIG. 6.

In one embodiment, a single 3D convolutional layer is employed as the temporal module. In the examples disclosed herein, the importance of selecting the right temporal module is demonstrated by comparing it to two naive approaches: element-wise average and element-wise maximum.

Inserting learned temporal modules at each skip connection level is motivated by the fact that different types of temporal information may useful for higher level versus lower level features.

In some embodiments, the choice of loss function has a significant impact on the quality of generated results. It is known that pixel-wise losses such as Mean Squared Error (MSE) and mean absolute error (MAE) correlate poorly with perceived image quality. On the other hand, these loss functions have been found to correlate better with human perception, when they are computed in a perceptually-appropriate representation space. In the latter case, these loss function are referred to as “perceptual losses”.

Three different loss functions are used in training U-Nets in the disclosed experimental examples: MSE, MAE, and a perceptual loss function. In some embodiments, the sum of the MAE's computed using the feature maps extracted from the first three blocks of VGG16 pretrained on the ImageNet dataset are used for the perceptual loss.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Experimental Setup

Below, the task of generating stereo video from 2D video is described, and a neural network-based approach is devised for this purpose. More specifically, a neural net architecture was designed suitable for turning a set of 2D frames into an alternative 2D view and a set of appropriate loss functions was selected.

“Stereo vision” as used herein refers to a pair of images (or video frames), corresponding to the left and right views of the same scene. The task of 2D-to-3D reconstruction, or equivalently stereo-video reconstruction, is defined as a problem of generating a missing view (either left or right) given the other (observed) view. Without loss of generality, the left view is taken as the observed input and the right view is used as the missing view to be reconstructed. In the case of video, multiple frames of the left view are available, while the availability of any right-view frame is not assumed.

More specifically, the systems and methods disclosed herein solve the task of online frame-level stereo-video reconstruction. Given a sequence of left-view frames x_{≤t} up to time t, the corresponding right view yₜ at time t is predicted.

Because a surgical video can be arbitrarily long, and also because it is unlikely that frames from long ago, which likely show different scenes, are useful, only a few frames K from the immediate past are considered as the input. In other words, a system solving this task takes the most recent K frames, x_{t-K:t}∈ [symbol] ^{K×3×H×W} and outputs the right view yₜ∈ [symbol] ^{3×H×W} where we assume three-channel frames of width W and height H.

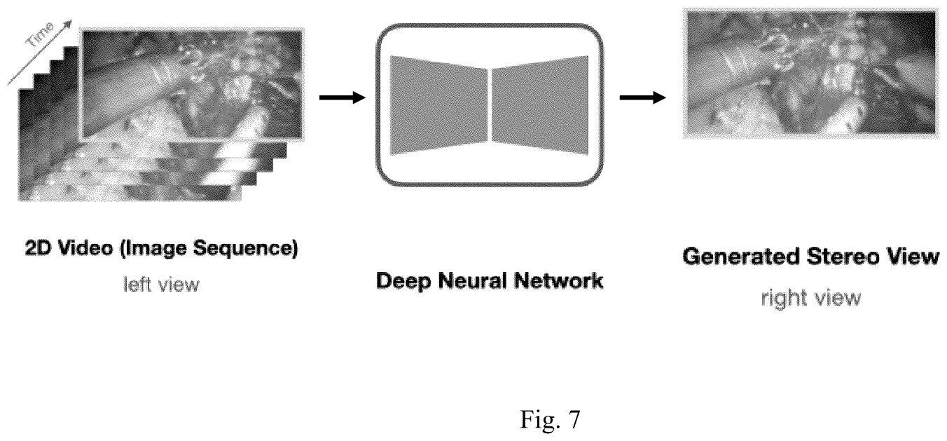

The task may be further described with reference to FIG. 7, which shows a model taking as input a variable number of consecutive video frames and predict a corresponding stereo view for the current (latest) frame at time t. Models are trained to minimize the reconstruction error of the right view, given the left view as input.

For training the 3D reconstruction models, the da Vinci endoscopic dataset from the Hamlyn Center for Robotic Surgery was used. The dataset consists of rectified stereo images (384×192 resolution) partitioned into 34,241 train, and 7,192 test sequences. The videos were captured in vivo during a partial nephrectomy procedure performed with a da Vinci surgical system. No data augmentation or transformations were applied. Using existing stereoscopic views to train the models served to minimize the reconstruction error between the generated and the true right view. No explicit depth information or intrinsic camera parameters were used or required for the methods disclosed herein.

The training setups differed, for example by varying the number of input frames and the network configuration of U-Net, to investigate the effect of using multiple frames for 2D-3D reconstruction. More specifically, the number of input frames was varied between one (current only), five, and ten consecutive frames. Five-and six-layer U-Nets were tested to understand whether more layers facilitate capturing temporal information.

All networks were trained with the Adam optimizer from scratch with the constant learning rate of 0.0001. Each model was trained on a single NVIDIA V100 GPU for no more than 30 hours. A mini-batch size of 16 was used although sometimes smaller mini-batches were used to work with the limited onboard memory, such as when a network with the 10-frame input was trained. 16 bit precision was used whenever possible in order to maximize efficiency with the limited onboard memory.

Two baseline algorithms were used for comparison. In both cases, the left view was shifted by a global disparity δ. The global disparity value δ was determined by minimizing the loss (MSE, MAE, or Perceptual) of the validation set. The algorithms differed in that the one fills the missing pixels with zeros (black) and the other copies the missing pixels from the input resulting in a duplicated strip on one side of the generated frame.

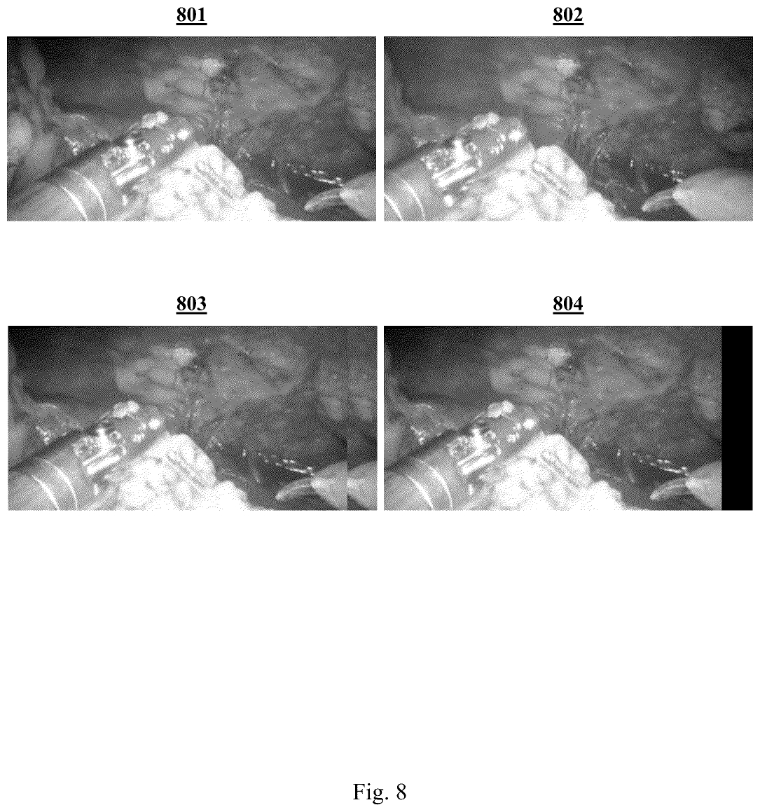

An example of the baseline algorithms is shown in FIG. 8. Image 801 is the left (input) view while image 802 shows the right (target) view used for training. Images 803 and 804 are baseline views, with image 803 using the original pixels in the missing region on the right side of the image, while image 804 uses a black strip.

Quantitatively assessing the quality of generated stereo video is itself an open problem. In much of the disclosed evaluation, the problem is simplified by assessing the image quality of generated stereo views, following the convention in previous work, rather than assessing full stereo video. Various automated metrics have been proposed to assess the quality of generated images and video clips, including structural similarity index (SSIM), the peak signal to noise ratio (PSNR), visual information fidelity (VIF), and Frechet inception distance (FID). Out of these metrics, a family of so-called perceptual metrics have been recently identified to be superior to the other metrics in how closely they correspond to human qualitative assessment. It is unclear and has not been established whether these metrics are suitable in the domain of endoscopic surgery. As an initial attempt at establishing the suitability of automated metrics in the domain of endoscopic surgery, a diverse set of automated metrics were correlated against human-perceived quality obtained via a series of reader studies with ten faculty surgeons.

Designing measures for the perceptual quality of generated images is an active area of research. The most widely used evaluation protocols for video rely on image similarity-based metrics. The disclosed examples measure the following five image quality metrics: (1) Peak signal-to-noise ratio (PSNR); (2) Structural Similarity Index (SSIM); (3) Fréchet Inception Distance (FID), (4) Learned Perceptual Image Patch Similarity (LPIPS) (with AlexNet backbone), and (5) Deep Image Structure and Texture Similarity (DISTS). All the metrics were studied to determine their correlation with human perception in the context of a surgical endoscopy video.

Two main reader studies were conducted. The first reader study focused on evaluating individual predictions from the U-Net variants. Each expert was asked to choose a better generated frame between two candidates. In the second study, the experts were asked to assess the quality of stereo-vision video using a virtual reality (VR) kit. Because of time constraints, a subset of promising models from the first reader study was used for 3D video quality assessment.

Eight models were selected for the frame-level reader study. The selection was based on a mix of automatic evaluation metrics and qualitative feedback from a supervising surgeon.

The eight models selected for the frame-level reader study are described in Table 1 below.

TABLE 1

| # | Model Description | Loss |
|---|---|---|
| 1 | Single Frame | Perceptual |
| 2 | Single Frame | MAE |
| 3 | Single Frame, 6 layers | MSE |
| 4 | 5 Frames | Perceptual |
| 5 | 5 Frames, 6 layers | Perceptual |
| 6 | 5 Frames, 6 layers | MSE |
| 7 | 5 Frames, upsampling: | Perceptual |
| | bilinear interpolation | |
| 8 | 10 Frames | MSE |

Unless specified otherwise, each model had five U-Net blocks and used transpose convolution for upsampling. If multiple input frames were used, a spatio-temporal U-Net architecture was used with 3D convolution temporal module.

These models were selected from an initial set of over 40 different configurations of architecture and loss as presented in Table 2 below. The top 10 models across four metrics were selected: LPIPS, FID, SSIM and PSNR as well as whether there were qualitative differences. Multiple rounds of evaluation were performed by a supervising surgeon to assess whether the generated images were visibly different and the surgeon's preference was ranked.

First, all spatio-temporal models that used a maximum or average temporal module were eliminated. The generated images created the effect of double vision when evaluated by human reviewers. Furthermore, there were no examples where a stacked multi-frame approach clearly outperformed using a single frame, thus none of those models were included. All selected multi-frame models therefore used a spatio-temporal architecture with the 3D convolution.

TABLE 2

| | |
|---|---|
| Loss Function | MSE, MAE, Perceptual |
| Temporal Structure | Single frame, stacking, spatio-temporal: |
| | 3D conv, max, avg |
| Number of input frames | 1, 5, 10 |
| U-Net blocks | 5, 6 |
| Upsampling mechanism | Transpose Convolution, Bilinear Interpolation |
| Sigmoid on the output | Yes, No |
| Extra skip connection | Yes, No |

It is worth noting that the only multi-frame models selected were those that used the spatio-temporal architecture, with a single 3D convolutional layer as the temporal module. Initial evaluation showed that stacking multiple frames resulted in similar, or worse, performance compared to using just a single frame. On the other hand, the spatio-temporal network with an average or maximum temporal module produced visible inconsistencies such as double vision.

To acquire a human perceptual assessment of the generated results at the frame level, a reader study was conducted for collecting and analyzing expert judgment. Data was gathered from 10 experts. The experts had between 6 and 30 years of medical experience, and all of them have experience performing endoscopic surgery.

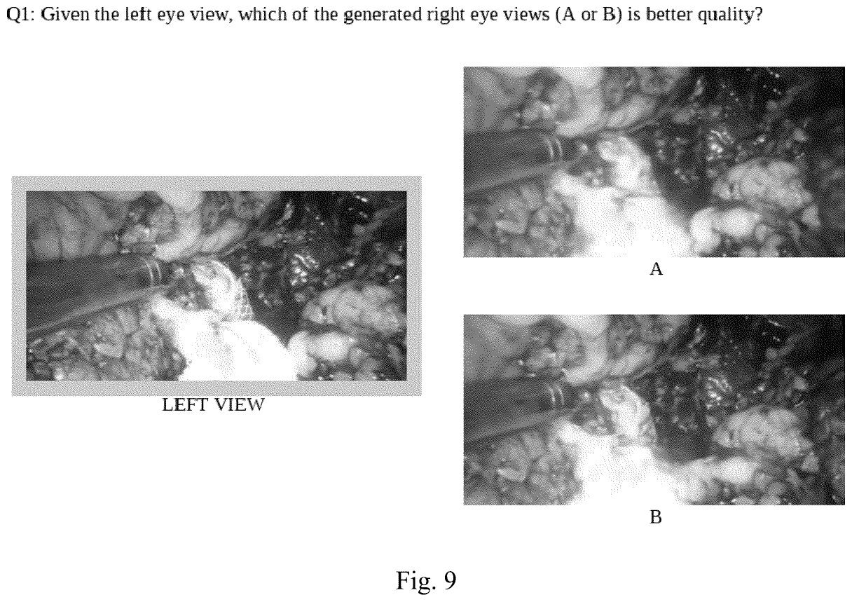

The two-alternative forced choice (2AFC) method was used to compare the eight different models. Each model was compared against every other model three times, using the same three examples. Additionally, each model was compared against the target once (based on the same image). This resulted in a total of 92 ((₂⁸)×3+8) comparisons. In a given trial, experts were shown two frames generated by two distinct models (candidate synthetic right-view frames) as well as the corresponding left-view frame used as an input to the models. Experts were then asked the following question: Given the left view, which of the generated right views (A or B) is of better quality? Each image was shown in the native resolution of the dataset (384 192 pixels), while the experts had an option to zoom in on any portion of the presented frame. There was no time limit for each trial. An example is shown in FIG. 9.

The Bradley-Terry model was employed to convert pair-wise comparison results to global rankings. Given a set of models to be ranked, the probability of selecting the output from some model i from a presented pair of outputs from model i and j is

$$P ( i ) = \frac{{α}_{i}}{{α}_{i} + {α}_{j}}$$

where αᵢ is the worth of item i. The PlackettLuce package was used, which reduces to the Bradley-Terry model given a list of pairwise-comparisons, to estimate relative rankings of the reader study models. Worth and bounded quasi-standard errors were estimated for each model. Because all models compared were expected to have worth greater than 0, worth was transformed to the log scale to provide bounds on confidence intervals such that they are constrained to be greater than 0. The target images were chosen as the reference such that the quasi-standard errors for the other models could be estimated.

A video-level reader study was then performed. The quality of an individual frame is not a sufficient proxy for stereo video quality, for a variety of reasons. A successful stereo video reconstruction requires that the viewer's perception of 3D depth remains consistent across consecutive frames. The shift between the two views that form a stereo pair is subtle. It is difficult to assess from a single left frame whether a given right frame would in fact result in realistic 3D depth perception when viewed in stereo. Given only a single stereo pair, it would be impossible to tell whether the model applies consistent transformation for reconstruction across time.

Five models were selected from the first reader study and further evaluated using a VR kit. Experts were first presented with a 25-second 2D clip. They then watched the same clip however this time in 3D generated from one of the selected models. The 3D videos were played using a Google Cardboard VR headset with an iPhone 12. Experts were then asked (1) whether the 3D video provided a better viewing experience than the 2D video did, and (2) to rate the quality of the reconstructed 3D video on the scale from 1 (worst) to 5 (best).

### Results

A major finding from the first reader study was that the proposed U-Net variant benefits from the availability of multiple left-view frames as the input. This was clear from the top-three entries in Table 3 below according to both log-worth as well as the win rate. All three models took as input five left-view frames. This observation confirmed the earlier hypotheses that (1) multiple frames from one view facilitate reconstructing the other view, and (2) the proposed variant of the U-Net is capable of exploiting such temporal information from multiple frames.

Another finding was the importance of using a perceptual loss function for training these U-Nets. The top-5 models were all trained to minimize the perceptual loss based on VGG-16, while the rest of the models were trained with either MSE or MAE in the original pixel space. This finding is in line with earlier observations on the importance of using a perceptual loss for training image generation models.

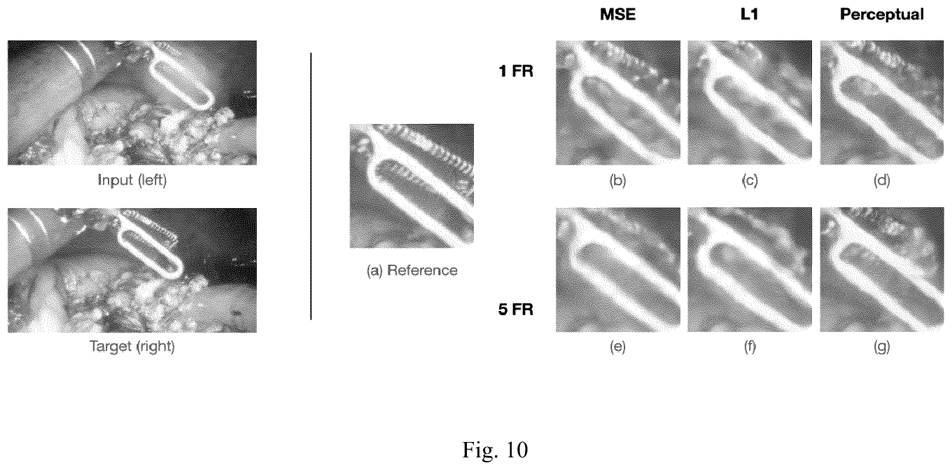

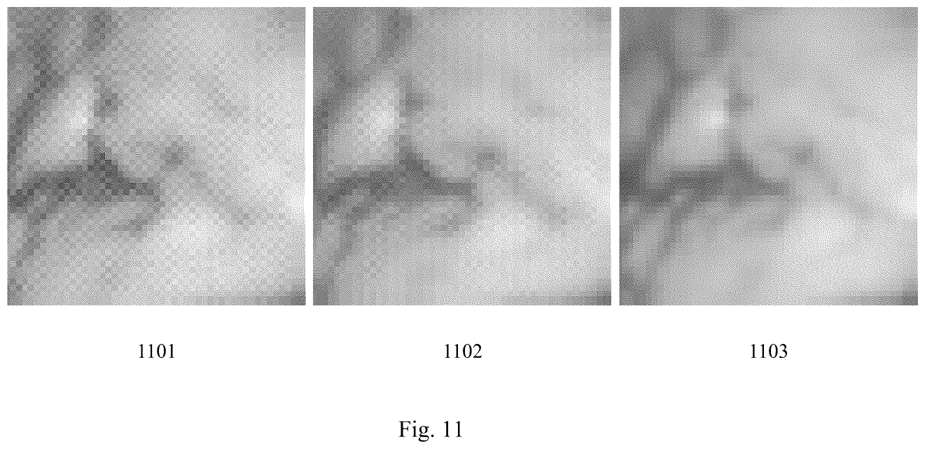

Qualitatively, models trained with perceptual loss generate markedly sharper results, as shown in FIG. 10. However on close inspection, in some images a checkerboard-like pattern of artifacts is visible (see e.g. FIG. 11). In some situations, bilinear interpolation visibly reduces artifacts on the generated images.

TABLE 3

| | | Reader Study | | Automated Metrics | | | | |
|---|---|---|---|---|---|---|---|---|
| Rank | Model | log-worth | win % | LPIPS ↓ | DISTS ↓ | FID ↓ | PSNR ↑ | SSIM ↑ |
| 1 | 5 fr + perceptual + 6 layers | 0.00 ± 0.20 | 100 | 0.116 | 0.110 | 50.53 | 22.88 | 0.627 |
| 2 | 5 fr + perceptual | −0.91 ± 0.17 | 86 | 0.119 | 0.116 | 48.42 | 22.77 | 0.616 |
| 3 | 5 fr + perceptual + bilinear | −1.85 ± 0.16 | 52 | 0.124 | 0.117 | 53.68 | 23.25 | 0.710 |
| 4 | 1 fr + perceptual | −2.04 ± 0.16 | 52 | 0.125 | 0.119 | 52.56 | 22.63 | 0.624 |
| 5 | 10 fr + perceptual | −2.41 ± 0.16 | 38 | 0.131 | 0.120 | 50.16 | 22.63 | 0.620 |
| 6 | 1 fr + MSE + 6 layers | −2.55 ± 0.16 | 48 | 0.156 | 0.143 | 65.78 | 23.04 | 0.700 |
| 7 | 1 fr + MAE | −3.74 ± 0.20 | 19 | 0.156 | 0.140 | 74.66 | 23.14 | 0.716 |
| 8 | 5 fr + MSE + 6 layers | −4.42 ± 0.23 | 5 | 0.159 | 0.143 | 66.28 | 23.78 | 0.722 |

Table 3 shows a comparison of reader study results and the corresponding validation performance for five automated metrics. Model rankings and the log-worth scores were determined by the Bradley-Terry model. Each of the eight selected models are compared a total of 24 times. For each model, the percentage of times it wins the majority vote was calculated and recorded in the column “% win”. Note, the top model according to the qualitative rankings won the majority vote among ten surgeons in all 24 comparisons. The worst ranked qualitative model only won one out of 24 comparisons (5%). LPIPS and DISTS were the only two automated metrics that correctly identified both the best and worst models according to expert evaluation.

TABLE 4

| | Expert | LPIPS | DISTS | FID | SSIM | PSNR |
|---|---|---|---|---|---|---|
| Expert | 1.0 | 0.95 | 0.98 | 0.76 | 0.64 | 0.41 |
| LPIPS | | 1.0 | 0.98 | 0.71 | 0.67 | 0.52 |
| DISTS | | | 1.0 | 0.74 | 0.60 | 0.39 |
| FID | | | | 1.0 | 0.93 | 0.73 |
| SSIM | | | | | 1.0 | 0.88 |
| PSNR | | | | | | 1.0 |

Table 4 above shows the Spearman Rank Correlation Coefficient (SRCC) between subjective expert rankings and automated metric rankings. The SRCC was calculated using the rankings of eight models by ten experts (senior surgeons with endoscopy experience) and the rankings obtained from five automatic metrics.

From the five final columns of Table 3, it is evident that the recently proposed, advanced perceptual loss functions, such as DISTS and LPIPS, can choose the best models, according to the expert judgement. This is a positive finding in that in the future, DISTS and/or LPIPS may be relied on for rapid and cheap iteration in designing and finding better approaches to 2D-to-3D reconstruction in the context of surgical video. This will greatly reduce cost and increase the speed of innovation.

Unlike DISTS and LPIPS, MSE-based PSNR did not show any discriminative capability among these models, resulting in more or less similar scores for all the tested models. On the other hand, SSIM, which has been successful with natural images (not surgical images), ended up choosing the two worst models, suggesting major differences between natural and surgical images. FID did prefer the models trained with the perceptual loss, perhaps unsurprisingly because FID itself is a perceptual loss. FID however ended up favoring the pixel-shift baselines over any of learned models, which significantly limits the applicability and reliability of FID.

FID failed as a reliable measure in this study. Whereas all the other metrics ranked the naive baseline models poorly, the FID metric scored them better than any of the trained models. (see baseline results in Table 5 below). The images generated by the baseline models contain clear discontinuities: for example, a duplicated strip of the original image or a black strip in place of the missing pixels. (see FIG. 8) Because the baseline models are not candidate solutions to the task of 2D-to-3D reconstruction, FID was found to be unsuitable in this context.

TABLE 5

| | Pixel | | | | | |
|---|---|---|---|---|---|---|
| Baseline Model | Shift | DISTS | LPIPS | FID | SSIM | PSNR |
| Copy pixels, | −31 | 0.129 | 0.201 | 30.079 | 0.493 | 17.675 |
| perceptual loss | | | | | | |
| Copy pixels, MSE loss | −37 | 0.128 | 0.205 | 32.648 | 0.486 | 17.728 |
| Copy pixels, MAE loss | −37 | 0.128 | 0.205 | 32.648 | 0.486 | 17.728 |
| Fill zeros, | −384 | 0.800 | 0.840 | 549.658 | 0.002 | 5.254 |
| perceptual loss | | | | | | |
| Fill zeros, MSE loss | −32 | 0.145 | 0.237 | 35.148 | 0.458 | 16.067 |
| Fill zeros, MAE loss | −33 | 0.146 | 0.237 | 35.655 | 0.455 | 16.043 |

These observations were further confirmed by the rank correlations among automatic metrics and experts, as shown in Table 4. The ranking of the models by both DISTS and LPIPS correlated almost perfectly with that of the experts. This correlation broke down quite rapidly with the other automatic metrics.

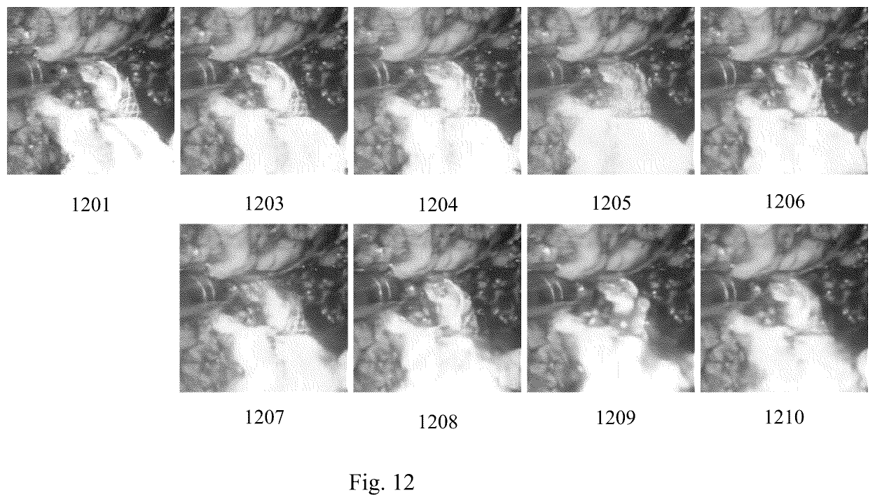

In order to get a better sense of where this difference among the models comes from, a few frames were manually inspected. FIG. 12 shows a representative example, displaying cropped tiles from the generations from all eight models. Image 1201 is the target right view, while images 1203-1210 are the model results ordered by ranking from best (1203) to worst (1210). The models trained using the perceptual loss produce visibly sharper images. One can easily discern visual details in the gauze in (1203-1207) than in the examples (1208-1210), the latter of which were produced by the only three models that did not use the perceptual loss.

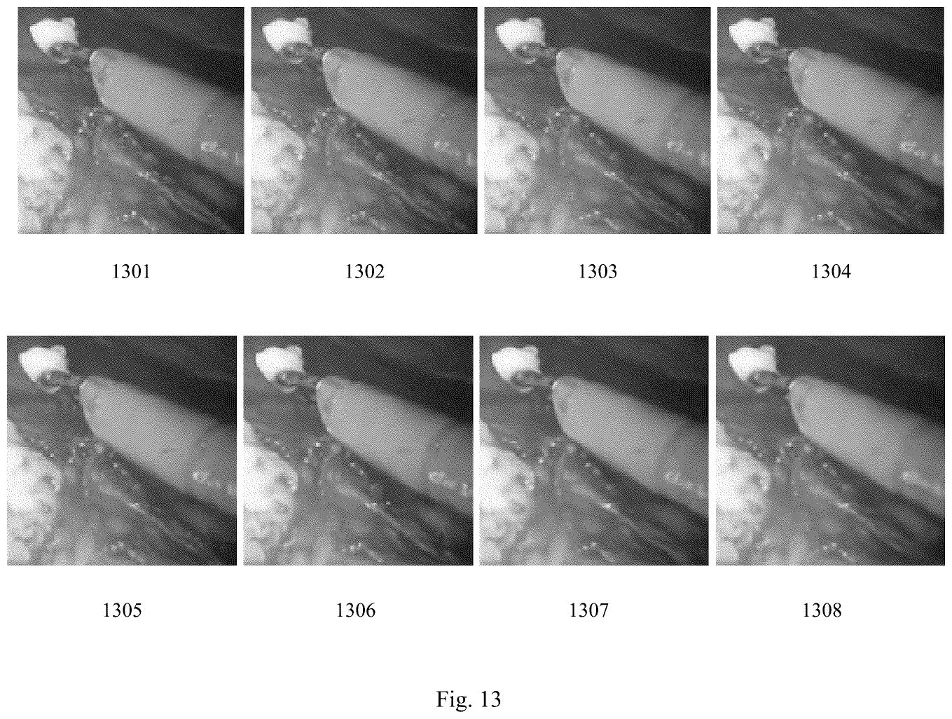

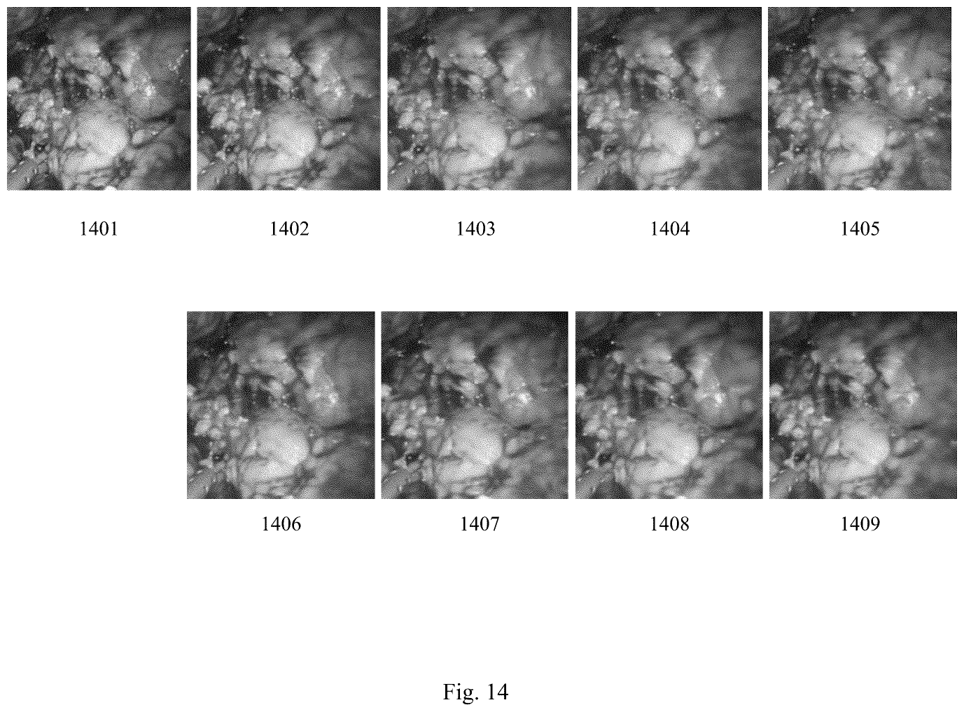

Additional results are shown in FIGS. 13 and 14. With reference to FIG. 13, eight cropped regions from the same image generated by different models are shown, ranked from best (1301) to worst (1308). With reference to FIG. 14, a cropped region of the target image is shown in image 1401, with eight generated images from different models shown in 1402-1409, ranked from best (1402) to worst (1409).

As additional analysis, another frame-level reader study was conducted with 10 non-expert readers who had no medical expertise. When the majority vote outcome was considered (for each pair of generated right-view images), the non-expert judgement agreed with the expert judgement approximately 80% of the time. This high-level agreement was attributed to visual anchors common for both expert and non-expert readers, such as the legibility of text or the sharp edges of a surgical tool. The disagreeing portion may have been caused by certain features that are visually subtle but that impact surgical outcomes and were therefore only detected by surgeons (expert readers) who are trained to exhaustively search for their presence. For instance, two surgeons commented on the appearance of the vasculature in the generated images. This is a fine detail that non-experts are unlikely to notice, but it is of critical importance to surgeons.

Despite this high level of agreement, a discrepancy was found between non-expert and expert judgements in the within-group disagreement rate. The average within-group disagreement was slightly higher among non-experts (0.26) than among experts (0.17), where the within-group disagreement was calculated as the percentage of members who disagreed with the majority vote of the group, averaged over all questions. This lower within-group disagreement rate among the experts may have been due to their attention to surgically relevant details that are not noticed nor taken into account by non-expert readers.

Based on this comparison between expert and non-expert readers, it can be concluded that one cannot fully substitute expert judgment with non-expert judgement. Nevertheless, reasonably high agreement indicates that a non-expert reader study together with automatic metrics, such as DISTS and LPIPS, can be used to iterate more rapidly and cheaply for future work.

The top-3 models from Table 3 were chosen in addition to the best MSE model and the best MAE model to form a group of five models to be tested in the second reader study. Two surgeons (expert readers) were recruited for this reader study.

The first finding from this reader study was that both readers almost always favored the 3D re-constructed surgical video clips over the corresponding 2D clips. This confirms the validity and importance of the proposed task of 2D-to-3D reconstruction in surgical video, and also demonstrates the effectiveness of the proposed U-Net based approach to this problem.

TABLE 6

| Model description | Avg. | StdDev | Comments |
|---|---|---|---|
| 5 fr, perceptual, | 4.67 | 0.52 | “Perfect.” |
| bilinear | | | “Great.” |
| 5 fr, perceptual | 3.83 | 0.55 | “Really like this one” |
| | | | “Temporal inconsistencies in grasper |
| | | | with shearing between frames” |
| 5 fr, perceptual, | 3.50 | 0.98 | “Loves it in 3D. Surprised at how |
| 6 layers | | | much liked it.” |
| | | | “Looked good. Couldn't see text on |
| | | | instrument.” |
| 1 fr, MSE, | 2.83 | 0.98 | “Periphery is not as crisp.” |
| 6 layers | | | “Right periphery is not very good.” |
| | | | “Blurrier, little disorienting.” |
| | | | “Globally less sharpness. Less details. |
| | | | Less pronounced stereoscopic effect” |
| 1 fr, MAE | 2.83 | 0.75 | “Depth perception was not as good |
| | | | as the first round.” |
| | | | “Blurrier, still pretty good.” |
| | | | “Not as detailed.” |

Table 6 above shows the results of the model evaluation in VR. The quality of each 3D video was scored on a scale of 1 to 5, where 5 was the highest quality. The table shows the average score and standard deviation for each model, as well as the surgeons' comments about each model.

A second finding confirms an observation from the first, frame-based reader study. The models trained with the perceptual loss function were favoured over the ones trained with either MSE or MAE, with significant margins. Based on the comments collected from the readers, one major cause behind this distinction is the blurriness associated with pixel-wise losses such as MSE or MAE. In particular, two of the comments regarding the MSE-trained model mention a lack of detail in the right periphery.

The favorite model among the experts in the second reader study was the third model from the first, frame-level reader study. The major difference between this model and the other two models trained with perceptual loss is that it uses bilinear interpolation for upsampling in the decoder rather than transposed convolution. In order to study the difference between these two upsampling implementations, the generated frames from two models were inspected manually (one with bilinear upsampling and the other with transposed convolution.) This revealed that transposed convolution tended to amplify a checkerboard artifact, shown in FIG. 11, similarly to earlier observation by Odena et al. Although the artifact is not as noticeable when individual frames are viewed independently, it may lead to a more disorientating visual experience when viewed in 3D, which results in experts preferring the model with bilinear interpolation.

With reference to FIG. 11, visual artifacts resulting from transposed convolutional layers in the decoder are shown. All three models use five frames, 3D convolution as the temporal module, and perceptual loss. Models differ only in the upsampling layers. 1101 and 1102 used transposed convolution, with six and five layers respectively, while 1103 used five layers of bilinear interpolation.

Overall, the second reader study once more confirmed the plausibility of the proposed task of 2D-to-3D reconstruction of surgical video captured by endoscopes.

### Conclusion

Endoscopy is a mainstay of modern minimally invasive surgery across multiple medical disciplines. Although 3D endoscopy provides depth perception, thereby creating a better viewing experience for surgeons, unlike more conventional 2D endoscopy, it is more challenging to use 3D endoscopy in practice. Disclosed herein is a method of converting 2D endoscopy video to 3D video, using modern deep learning. More specifically, a modified form of U-Nets is disclosed for generating a missing (right) view given a series of consecutive left-view frames, and its effectiveness was tested by running an extensive set of reader studies, both frame-level and video-level, together with experienced surgeons.

The reader studies along with thorough analysis have revealed a few major findings. First, expert readers preferred a generated stereo-vision video over the corresponding 2D video, confirming the usefulness of the proposed task and the effectiveness of the proposed deep learning based solution to it. Second, the models that were favored by expert surgeons were the ones that were trained with a perceptual loss, were given access to multiple consecutive frames of an observed view and were equipped with a convolutional temporal module. This finding verifies a hypothesis that temporal information, underlying multiple frames of one view, is critical to reconstruct the missing view and shows that the proposed variant of a U-Net is able to exploit such information from input frames. Third, two perceptual loss functions, DISTS and LPIPS, were identified that correlate well with expert judgements, which will enable rapid iteration in the future to improve algorithms for solving the proposed task without relying on time-consuming and costly reader studies. Finally, the above experimental examples demonstrate the importance of expert readers over non-expert (ones without any medical experience) readers in assessing 2D-to-3D reconstruction algorithms for surgical video. Overall, these findings indicate that the proposed task is feasible and useful, and that the proposed approach is promising and can be readily applied if needed.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### Cited References

The following publications are incorporated herein by reference in their entireties:
R Atul Kumar, et al. Stereoscopic visualization of laparoscope image using depth information from 3d model. Computer Methods and Programs in Biomedicine, 113(3):862-868, 2014.
Augustus Odena, et al. Deconvolution and checkerboard artifacts. Distill, 2016.
Benjamin Ummenhofer, et al. Demon: Depth and motion network for learning monocular stereo. CoRR, abs/1612.02401, 2016.
Christoph Fehn. Depth-image-based rendering (DIBR), compression, and transmission for a new approach on 3D-TV. In Mark T. Bolas, et al., editors, Stereoscopic Displays and Virtual Reality Systems XI, volume 5291, pages 93-104. International Society for Optics and Photonics, SPIE, 2004.
David Firth. 1. overcoming the reference category problem in the presentation of statistical mod-els. Sociological Methodology, 33(1):1-18, 2003.
Diederik P. Kingma and Jimmy Ba. Adam: A method for stochastic optimization, 2017.
Faisal Mahmood and Nicholas J. Durr. Deep learning and conditional random fields-based depth estimation and topographical reconstruction from conventional endoscopy. Medical Image Analysis, 48:230-243, 2018. ISSN 1361-8415.
Faisal Mahmood and Nicholas J. Dun. Deep learning-based depth estimation from a synthetic endoscopy image training set. In Elsa D. Angelini and Bennett A. Landman, editors, Medical Imaging 2018: Image Processing, volume 10574, pages 521-526. International Society for Optics and Photonics, SPIE, 2018.
G. Berci and K. A. Forde. History of endoscopy: What lessons have we learned from the past? Surgical endoscopy, 14(1):5-15, 01 2000.
Heather L. Turner, et al. Modelling rankings in R: The PlackettLuce package. Computational Statistics, 35:1027-1057, 2020.
J. Deng, et al. ImageNet: A Large-Scale Hierarchical Image Database. In CVPR09, 2009.
Jamie Gompel, et al. Field of view comparison between two-dimensional and three-dimensional endoscopy. The Laryngoscope, 124, 02 2014.
Juhyeon Kim and Young Min Kim. Novel view synthesis with skip connections. In 2020 IEEE International Conference on Image Processing (ICIP), pages 1616-1620, 2020.
Junyuan Xie, et al. Deep3d: Fully automatic 2d-to-3d video conversion with deep convolutional neural networks, 2016.
Karen Simonyan and Andrew Zisserman. Very deep convolutional networks for large-scale image recognition, 2015.
Kenan Koc, et al. The learning curve in endoscopic pituitary surgery and our experience. Neurosurg. Rev., 29(4):298-305; discussion 305, October 2006.
Keyan Ding, et al. Comparison of full-reference image quality models for optimization of image processing systems. International Journal of Computer Vision, 129(4):1258-1281 January 2021. ISSN 1573-1405.
Keyan Ding, et al. Image quality assessment: Unifying structure and texture similarity. IEEE Transactions on Pattern Analysis and Machine Intelligence, page 1-1, 2020. ISSN 1939-3539.
Khan W Li, et al. Neuroendoscopy: past, present, and future. Neurosurg. Focus, 19(6):E1, December 2005.
Leida Li, et al. Depth image quality assessment for view synthesis based on weighted edge similarity. In Proceedings of the IEEE/CVF Conference on Computer Vision and Pattern Recognition (CVPR) Workshops, June 2019.
Martin Heusel, et al. Gans trained by a two time-scale update rule converge to a local nash equilibrium, 2018.
Mehmet Turan, et al. Sparse-then-dense alignment-based 3d map reconstruction method for endoscopic capsule robots. Machine Vision and Applications, 29(2):345-359, 2018.
Mei Han and Takeo Kanade. A perspective factorization method for euclidean reconstruction with uncalibrated cameras. The Journal of Visualization and Computer Animation, 13(4):211-223,2002.
Menglong Ye, et al. Self-supervised siamese learning on stereo image pairs for depth estimation in robotic surgery, 2017.
Nahian Siddique, et al. U-net and its variants for medical image segmentation: theory and applications, 2020.
Olaf Ronneberger, et al. U-net: Convolutional networks for biomedical image segmentation, 2015.
Olivia Wiles, et al. Synsin: End-to-end view synthesis from a single image, 2020.
Paris P Tekkis, et al. Evaluation of the learning curve in laparoscopic colorectal surgery: comparison of right-sided and left-sided resections. Ann. Surg., 242(1):83-91, July 2005.
Ping Li, et al. On creating depth maps from monoscopic video using structure from motion. January 2008.
Radu Sibechi, et al. Exploiting temporality for semi-supervised video segmentation, 2019.
Ralph Allan Bradley and Milton E Terry. Rank analysis of incomplete block designs: I. the method of paired comparisons. Biometrika, 39(3/4):324-345, 1952.
Richard Zhang, et al. The unreason-able effectiveness of deep features as a perceptual metric, 2018.
O. Ronneberger, et al., (2015) U-Net: Convolutional Networks for Biomedical Image Segmentation. In: N. Navab, et al. (eds) Medical Image Computing and Computer-Assisted Intervention—MICCAI 2015. MICCAI 2015. Lecture Notes in Computer Science, vol 9351.
Rui Song, et al. MCL-3D: a database for stereoscopic image quality assessment using 2d-image-plus-depth source. CoRR, abs/1405.1403, 2014. URL http://arxiv.org/abs/1405.1403.
Salvatore Livatino, et al. Stereoscopic visualization and 3-d technologies in medical endoscopic teleoperation. IEEE Transactions on Industrial Electronics, 62(1):525-535, 2015.
Sergiu Oprea, et al. A review on deep learning techniques for video prediction. IEEE Transactions on Pattern Analysis and Machine Intelligence, page 1-1, 2020. ISSN 1939-3539.
Stefan Petscharnig and Klaus Schöffmann. Learning laparoscopic video shot classification for gynecological surgery. Multimedia Tools and Applications, 77(7):8061-8079 April 2018.
Xingtong Liu, et al. Self-supervised learning for dense depth estimation in monocular endoscopy. CoRR, abs/1902.07766, 2019. URL http://arxiv.org/abs/1902.07766.
Xingtong Liu, et al. Self-supervised learning for dense depth estimation in monocular endoscopy. In Danail Stoyanov, et al., editors, OR 2.0 Context-Aware Operating Theaters, Computer Assisted Robotic Endoscopy, Clinical Image-Based Procedures, and Skin Image Analysis, pages 128-138, Cham, 2018.
Zhou Wang, et al. Image quality assessment: From error visibility to structural similarity. IEEE Transactions on Image Processing, 13:600-612, 2004.

What is claimed is:

1. A system for generating a target image, comprising:
an endoscope having an image collection component;
a computing device communicatively connected to the image collection component of the endoscope, comprising a non-transitory computer-readable medium with instructions stored thereon, which when executed by a processor perform steps comprising:
receiving at least one input image from the image collection component of the endoscope;
providing the at least one input image as an input to a machine learning algorithm;
generating a target image from the at least one input image using the machine learning algorithm; and
providing the at least one input image and the target image to a display driver; and
a display device, communicatively connected to the computing device, and configured to display the images provided to the display driver.

2. The system of claim 1, wherein the at least one input image comprises a sequence of at least five frames of a video recorded by the image collection component.

3. The system of claim 1, wherein the image collection component is a camera.

4. The system of claim 1, the endoscope further comprising a tube with the image collection component positioned at a distal end of the tube, the tube having an outer diameter of at most 10 mm.

5. The system of claim 1, wherein the computing device is positioned in the display device.

6. The system of claim 1, wherein the computing device is positioned in the endoscope.

7. The system of claim 1, wherein the machine learning algorithm is selected from a convolutional neural network, a generative/adversarial neural network, or a U-Net.

8. The system of claim 1, the steps further comprising buffering a sequence of input images to process with the machine learning algorithm.

9. The system of claim 8, wherein the sequence comprises at least five input images.

10. A method of training a machine learning algorithm for 3D reconstruction, comprising:
providing a set of rectified stereo video frames;
selecting a training subset of the set of rectified stereo video frames and isolating one view from each of the selected stereo video frames;
providing a sequence comprising at least one input video frame from the isolated view to a machine learning algorithm to generate a target frame corresponding to the at least one input video frame;
calculating a loss function value from the generated target frame by comparing it to the known corresponding video frame from the set of stereo video frames; and
adjusting at least one parameter of the machine learning algorithm based on the calculated value of the loss function.

11. The method of claim 10, wherein the sequence comprises at least five video frames.

12. The method of claim 10, wherein the machine learning algorithm is selected from a convolutional neural network, a deep neural network, a U-Net, or a generative/adversarial neural network.

13. The method of claim 10, wherein the loss function is selected from mean-squared error, least absolute deviations, least square errors, or perceptual loss function.

14. The method of claim 10, wherein the machine learning algorithm comprises an automated metric selected from Learned Perceptual Image Patch Similarity, Deep Image Structure and Texture Similarity, Frechet Inception Distance, Peak signal-to-noise ratio, or Structural Similarity Index.

15. The method of claim 14, wherein the automated metric is selected from Learned Perceptual Image Patch Similarity and Deep Image Structure and Texture Similarity.

16. A method of generating a stereoscopic image for a user of an endoscope, comprising:
receiving at least one input image from an image collection component of an endoscope;
providing the at least one input image as an input to a machine learning algorithm;
generating a target image from the at least one input image using the machine learning algorithm; and
displaying the at least one input image and the target image on a display device as a stereoscopic image.

17. The method of claim 16, wherein the at least one input image comprises a sequence of at least five frames of a video recorded by the image collection component.

18. The method of claim 16, wherein the machine learning algorithm is selected from a convolutional neural network, a generative/adversarial neural network, or a U-Net.

19. The method of claim 16, further comprising buffering a sequence of input images to process with the machine learning algorithm.

20. The method of claim 19, wherein the sequence comprises at least five input images.

21. The method of claim 16, further comprising upsampling the at least one input image using bilinear interpolation or strided transpose convolution.
